(19) United States
(12) Patent Application Publication
(10) Pub. No.: US 2007/0067010 A1
(43) Pub. Date: Mar. 22, 2007

(54) CENTER_HOLLOWED_TUBULAR_SHAPED BALLOON FOR ANGIOPLASTY PROCEDURES

(76) Inventors: William Tianyuan Wang, Las Vegas, NV (US); Zhiyong Wang, Las Vegas, NV (US)

Correspondence Address: William T. Wang;Z Wang, 688 Homewillow Ave, Las Vegas, NV, 89123, US

(21) Appl. No.: 11/162,784

(22) Filed: Sep. 22, 2005

(51) Int. Cl. A61F 2/06

(52) U.S. Cl. 623/1.11; 606/194

(57) ABSTRACT

The present invention is directed to a procedure and a special balloon used in balloon angioplasty and stent installation in percutaneous transluminal coronary angioplasty (PTCA), which employs a heart catheter with a balloon at its distal end. After being inflated, the balloon is in doughnut shape with a hole in the center; its length is made to fit the length of the stent to be inflated. During an angioplasty procedure, the balloon is pushed forward into the stenosis and inflated with a device to dilate the blockage. The doughnut_shaped balloon provides a pass way for blood to flow through the coronary artery vessel, which does not cause angina akin to a heart attack resulted from traditional tube_shaped balloons used in the PTCA procedure that completely block blood flow after being fully inflated during balloon angioplasty and stent installation.

### TECHNICAL FIELD OF THE INVENTION

The present invention relates generally to angioplasty balloon, and more specifically is concerned with a doughnut shaped balloon which allows blood flow through its center hole during a PTCA procedure or other types of internal body application.

### PRIOR ART

Developed in the late 1970s, high-pressure balloons have been used in angioplasty, a procedure that opens blood vessels clogged by built-up and fatty plaque with a specially made balloon, which is tightly wrapped around a catheter shaft to minimize its profile. The balloon is inserted into the patient's blood vessels to the site of the narrowed section, then being inflated typically with a radiopaque solution or saline forced through a syringe, exerts high pressure, which compresses the plaque against the wall of blood vessel to reopen the clogged area. For retraction, a vacuum is pulled through the balloon to collapse it. The procedure was developed as a less invasive and less costly alternative to coronary bypass.

High-pressure balloons are now used in a wide range of diagnostic and therapeutic devices due to improvements in materials, balloon design and fabrication technology [U.S. Pat. No. 4,351,341; U.S. Pat. No. 4,824,436; U.S. Pat. No. 4,906,244; U.S. Pat. No. 6,746,425 B1]. These improvements include increased diameters, additional lengths, ultra_thin walls (for minimal invasion and a smaller profile), varying diameters throughout the balloon, custom shapes, tapered ends and angles, and specialty coatings [U.S. Pat. No. 4,909,252; U.S. Pat. No. 4,994,033; U.S. Pat. No. 5,342,301]. By 2005, over 100 designs have been patented in the U.S. for balloon angioplasty.

The first angioplasty balloons were fabricated from flexible polyvinyl chloride (PVC). They were relatively thick_walled and low_pressure compared to today's high_pressure balloons. Cross_linked polyethylene came into use in the early_to mid_{—}80s, about the same time that polyester (PET) polyethylene terephthalate was adopted for high_pressure balloons. Those two materials replaced PVC to a large degree. Nylon balloons came out in the late 1980s, and polyurethane balloons followed in the early 1990s. Nylon, while not as strong as PET or as compliant as PET, was seen as a compromise because it was softer than PET, but relatively thin and relatively strong. Today most high_pressure medical balloons are made from either PET or nylon. PET offers advantages in tensile strength, and maximum pressure rating while nylon is softer.

For angioplasty, balloons must have a controlled or repeatable size (diameter vs. pressure) in order to ensure that the balloon will not continue to expand and damage or rupture the artery after it opens the blockage. Balloon compliance is the term used to describe the degree to which a high_pressure balloon's diameter changes as a function of pressure. A low_compliance, high_pressure balloon might expand only 5_{—}10% when inflated to the rated pressure while a high_compliance, high_pressure balloon might stretch 18_{—}30%.

Rated pressures for angioplasty balloons are typically in the range of 2_{—}20 atmospheres (30 to nearly 300 psi) depending on the size; the larger the diameter, the lower the rated pressure. This is due to the fact that as the diameter of a balloon increases, the stress in the balloon wall increases when inflated to its nominal diameter. One major advantage of PET is its unusual ability to be molded into ultra thin walls and very precise shapes. Since PET is ultra_thin_walled, ranging from 5 to 50 microns (0.0002″ to 0.002″), it is capable of producing balloons of extremely low profile. High_pressure PET balloons can be produced with diameters from 0.5 mm to 50 mm or more, in any working length, while maintaining very thin walls. They can be custom designed with varying diameters along the length of the balloon and tapered ends from 1 to 90 degrees. Other benefits include excellent heat transfer characteristics and optical clarity, making PET balloons suitable for use with Nd: YAG and other lasers, ultrasound and microwave energy.

Nylon high_pressure balloons are softer than PET balloons, although not as strong, thus requiring a thicker wall for a given burst pressure. This generally means that nylon balloons will have a larger profile than PET upon insertion into the body and crossing a lesion, but because the material is softer, it is more easily refolded, thus making it easier to withdraw into the guiding catheter or introducer sheath.

Angioplasty balloons may be formed in various sizes ranging from small coronary size balloons to large diameter balloons used in peripheral arteries.

Balloons may also be formed with different cone angles to meet various balloon taper requirements.

Balloons are formed in a variety of sizes using high performance materials ranging from 2 to 25 mm in diameter. A small round balloon may be used in fallopian tube plasty while a large balloon may be used in valvuloplasty.

U.S. Patent Application Publication No. 20,010,008,976 of Wang, Lixiao published on Jul. 19, 2001, discloses a method for installing a stent in a vessel utilizing a single balloon catheter for both low pressure predilation at a relatively small diameter to open the lesion sufficiently to allow insertion and deployment of the stent across the lesion and for subsequent high pressure embedding of the stent in the vessel wall. The same balloon catheter may also be employed to insert and deploy the stent. The balloons utilized in the method have a stepped compliance curve which allows for predilation at a low pressure and predetermined diameter and for high pressure embedding at a substantially larger diameter. The balloons may be provided with a configuration in which only a portion of the balloon has a stepped compliance curve while a further portion has a generally linear compliance profile. The drawback of this approach is with such balloons is that the blood flow is interrupted causing heart attacks.

One prior art (U.S. Pat. No. 4,581,017 of Sahota) attempt to provide a catheter with small orifices in the proximal end adjacent to the balloon, these orifices provides a flow path for blood during the angioplasty process. However, due to the limited diameter of a catheter, the device of Sahota still results in insufficient cross sectional flow area in the blood vessel. To provide better blood flow, Goldberger (U.S. Pat. No. 4,909,252) disclosed a catheter utilizing a perfusion balloon, which has a donut-shaped cross section with a central opening for blood flow during a valvuloplasty or an angioplasty process. The double walled balloon is attached to a catheter side by side along its external wall and retained to the catheter with clips. The inner wall of the balloon is connected with external wall by ribs to keep it stay in place. Goldberger's invention may provide better blood flow for valvuloplasty or an angioplasty process. However, to make rib-connected double walled balloon at 2-3 mm in maximum diameter is a challenge to manufacturing industries. Also side by side connection of the balloon to a catheter not only increases the profile of the assembly, but also makes the assembly in irregular shape, which causes additional difficulty in delivering the balloon to the already narrowed stenotic region. In addition, hinging clips onto external surface of a catheter is not a desirable approach for procedures related to inter artery operations.

### SUMMARY OF THE INVENTION

The present invention provides a special angioplasty balloon used in stent installation in PTCA and other type of diagnostic and therapeutic procedures. The angioplasty balloon can be precisely folded in a small profile, and then fitted inside a stent. Next the stent is evenly crimped down around the balloon. Mounted on the end of a catheter, the balloon/stent is inserted into a blood vessel and remotely maneuvered into position by the physician. The application of doughnut_shaped balloon is not limited to PTCA; it can also be implemented to other types of diagnostic and therapeutic procedures. Therefore, the present invention provides a less invasive alternative to traditional PTCA procedure as will be apparent to those skilled in this art from a careful reading of this application including its claims.

### BRIEF DESCRIPTION OF THE DRAWINGS

In order to better understand the invention and to illustrate it in practice, non_limiting examples of some preferred embodiments will now be described, with reference to the accompanying drawings, in which:

FIG. 1 is a schematic illustration of the center_hollowed tubular shaped balloon after being inflated.

FIG. 2 is a schematic perspective transparent illustration of the center_hollowed tubular shaped balloon connected to a catheter at the both ends of the balloon.

FIG. 3 is a cross_sectional view of the assembly taken along line Q_Q.

### DETAILED DESCRIPTION OF THE PREFERRED EMBODIMENT

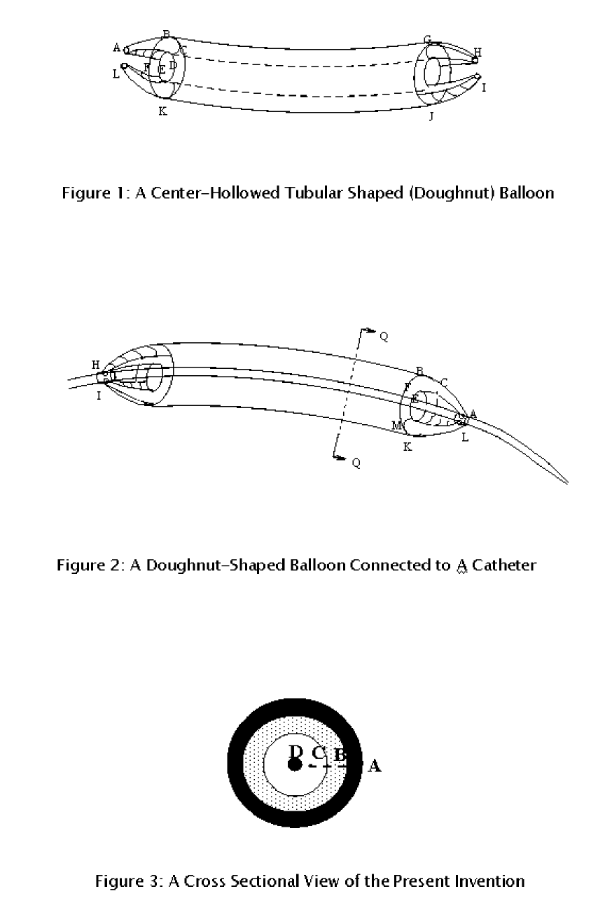

When the balloon is inflated, the main body of the balloon expands into center hollowed tubular shape (BGJK in FIG. 1). It looks like a doughnut with a hole in the center; its length is made to fit the length of the stent to be inflated. The two parallel dash lines represent the inner diameter of the balloon, which is also the pass way for blood to flow through. Tiny holes at A, H, I, and L are for radiopaque solution, or saline, or other media to flow into the balloon, inflating it into full size. The holes are also for retrieving radiopaque solution, or saline, or other media out of the balloon, causing it collapses. The outward pressure from the balloon re_expands the stent to force open the blockage. At each end of the balloon, there is an (or more than one) extension portion (such as ABCDEF in FIG. 1 and shape may vary) of the balloon connected to the main body of the balloon (BGJK in FIG. 1).

The extension portion is part of the thin_wall balloon with a hole at point A (also L in FIG. 1) which is tightly connected to a hole in the heart catheter at the distal end as seen in FIG. 2 of the Drawings. The balloon is fully inflated in FIG. 2. At the other end, the hole connects to the center_hollowed tubular shaped balloon by thin wall balloon material at junction BCDEF. There is no wall within the closed junction area BCDEF to separate them apart. Therefore, center_hollow wedge_shaped balloon, ABCDEF, is interconnected with the main body of BGJK (FIG. 1). Other than the connecting area(s) with the extension portion of the balloon, the center_hollow tubular shaped balloon is sealed by the inner layer (dash line in FIG. 1), external layer (BGJK outline in FIG. 1), and end layers (rings BCKF and GJ) of balloon thin walls.

Point H, as seen in FIG. 1 of the Drawings, possesses the same structure as wedge_shaped balloon ABCDEF, is connected to another point of the heart catheter as seen in FIG. 2 of the Drawings. At both points A and H there are holes in the catheter, which allow a radiopaque solution, or saline, or other type of media, typically in the range of 2_{—}20 atmospheres or 30 to nearly 300 psi, to inflate the balloon through hole A (and L, H, I) from the extension portions of the balloon to the main body of the balloon.

The wedge_shaped extension balloon is connected to the main body of the balloon with thin_wall balloon material, therefore, a radiopaque solution, or saline, or other type of media flow from hole A and L, H, I as seen in FIG. 2 of the Drawings, continuously to pump up the entire balloon. As the balloon main body inflates, its center hole opens up to allow blood flow through the inner hole from one end of the balloon to the other end. At both ends, gaps between the wedge_shaped extension balloon (ABCDEF and LKM for example) allow blood flow from the inner balloon hole to the artery or vise versa as seen in FIG. 2. The number of the wedge_shaped extension balloons at each end of the doughnut_shaped balloon may vary. The shape of the extension balloon may vary as well, it may be wedge_shaped as illustrated in FIG. 2, or may take different shapes.

A cross sectional view of the present invention is provided in FIG. 3, in which an imaginary plane cut perpendicularly to the axis of the main body of the doughnut_shaped balloon (coincide with the heart catheter, view point of Q_Q in FIG. 2). The external layer (AB) shown in FIG. 3 represents the artery wall thickness; the black dash_line circle at point B represents the stent opened up by the balloon; Point B inner layer also represents the external wall of the balloon and point C represents the inner wall of the balloon; from point C to D is the hollow center of the balloon which allows blood to flow through the inflated balloon; black dot D represents the diameter of the heart catheter.

After positioning the stent in the desired location, the balloon is pressurized to expand the stent securely against the arterial wall. The maximum inflation diameter is typically larger than the arterial diameter to establish good contact. The expansion step from the stent on the balloon in the delivery configuration to the maximum dimension at full pressure increases the stent diameter to its full size, and causes additional deformation in the device. Next, a vacuum is pulled through the angioplasty catheter to collapse the balloon back to a small profile, leaving the stent in place. The stent unloads elastically, typically reducing its diameter by 5_{—}10 percent from the maximum inflation diameter. From this point, the stent is deployed in the artery; the in_vivo loads from the body are applied. The angioplasty catheter can be withdrawn safely with the deflated balloon securely attached to points A and H.

The procedure is invented as a less invasive alternative to traditional PTCA procedure, which employs a tube_shaped balloon completely blocking blood flow after being fully inflated for approximately 2 minutes to press open the blockage and create a channel that increases blood flow through the artery. The traditional procedure causesáheart thump or skip and leads to angina akin to a heart attack because the artery is completely blocked while the balloon is inflated. Occasionally, the angioplasty balloon fails to deflate, which may cause serious injuries or even death to patients with the traditional PTCA procedure.

With the present invention, blood flow through the doughnut shaped angioplasty balloon during the PTCA procedure, which does not cause angina and prevents potential injuries or even death resulted from angioplasty balloon malfunction.

Materials for making the balloons can be polyvinyl chloride (PVC), cross_linked polyethylene (PE), polyester (PET), polyethylene terephthalate, Nylon, and others.

What is claimed is:

1. An angioplasty procedure of implementing a center_hollowed balloon (including its extension balloons on its both ends) for blood vessel enlargement and/or for stent installation in PTCA operation, comprising the steps of: (a) precisely fold a center_hollowed balloon (including its extension balloons on its both ends) in a small profile, and then fitted inside a stent; (b) the stent is evenly crimped down around the balloon, mounted on the end of a catheter; (c) one or more apperatures vertically located on the catheter connecting to the apperatures on one end of the extension balloons; (d) the other end of the extension balloon is interconnected to one end of a center_hollowed tubular shaped balloon; (e) the balloon/stent is inserted into a blood vessel and remotely maneuvered into position by the physician; (f) as radiopaque solution, or saline, or other type of media, being injected into the catheter, it flows from the hollow center of the catheter through its vertical apperatures into the extension balloon, then into the center_hollowed tubular shaped balloon to inflate it; (g) after the stent is installed, the radiopaque solution, or saline, or other type of media, being injected into the balloon are vacuumed out through the same channel to deflate the balloon; and (h) the balloon collapses into small profile tightly connected to the catheter, and then being removed out of blood vessel along with the catheter.

2. An angioplasty balloon comprising: (a) a center_hollowed tubular shaped balloon, and an (or more) extension balloon interconnected to the end of a center_hollowed tubular shaped balloon; (b) interconnected meaning that both parts are skin connected only with no physical barrier to separate them from within; (c) a center_hollowed tubular shaped balloon has an external surface wall, an internal surface wall, and connecting surface walls linking between both external and internal surface walls; (d) all walls are made of balloon materials; (e) radiopaque solution, or saline, or other type of media fills in the center_hollowed tubular shaped balloon between the external and internal surface walls to inflate the balloon; (f) as the balloon being inflated, the inner hole opens up to allow blood to flow through; (g) an (or more) extension balloon(s) interconnected to the end(s) of a center_hollowed tubular shaped balloon; (h) wherein the function of the extension balloon is to provide radiopaque solution, or saline, or other type of media, to flow from the catheter through the extension balloon to inflate the center_hollowed tubular shaped balloon, and then to deflate it and the extension balloon (s) by letting the radiopaque solution, or saline, or other type of media, to flow out of the center_hollowed tubular shaped balloon through the extension balloon back to the catheter; and (i) wherein the number of extension balloon connected to the center_hollowed tubular shaped balloon may vary.

3. An angioplasty catheter goes through the axis of a center_hollowed tubular shaped balloon. There is a hole (or more than one) vertically located on the catheter connecting to the holes on one end of the extension balloons. The other end of the extension balloon is interconnected to one end of a center_hollowed tubular shaped balloon. Before the balloon (both the extension and center_hollowed tubular shaped balloons) is inflated, it can be precisely folded in a small profile, and then fitted inside a stent. As a radiopaque solution, or saline, or other type of media, being injected into the catheter, it flows from the hollow center of the catheter through its vertical hole(s) into the extension balloon, then into the center_hollowed tubular shaped balloon to inflate it. After the stent is installed, the radiopaque solution, or saline, or other type of media, being injected into the balloon are vacuumed out through the same channel to deflate the main and extension balloons.

4. The application of center_hollowed balloons to other types of diagnostic and therapeutic procedures by using, such as, but not limited to, PTA catheters, Valvuloplasty catheters, Other dilation catheters, Stent delivery catheters, Heat transfer catheters, Photodynamic therapy (PDT), Laser balloon catheters, Cryogenic catheters, Drug delivery devices, Positioning catheters, Arthrectomy catheters, and so on.
